(19) United States
(12) Patent Application Publication
(10) Pub. No.: US 2009/0301234 A1
(43) Pub. Date: Dec. 10, 2009

(54) APPARATUS AND METHOD FOR MEASURING THE SURFACE FLUX OF A SOIL GAS COMPONENT

(75) Inventor: David Risk, Heatherton (CA)

Correspondence Address: BERESKIN AND PARR LLP/S.E.N.C.R.L., s.r.l., 40 KING STREET WEST, BOX 401, TORONTO, ON, M5H 3Y2, CA

(73) Assignee: ST. FRANCIS XAVIER UNIVERSITY, Antigonish, NS (CA)

(21) Appl. No.: 12/298,641

(22) PCT Filed: Apr. 27, 2007
(86) PCT No.: PCT/CA2007/000728

§ 371 Date: Feb. 6, 2009

Related U.S. Application Data
(60) Provisional application No. 60/795,164, filed on Apr. 27, 2006.

(51) Int. Cl. G01N 1/22; G01N 33/24

(52) U.S. Cl. 73/864.83

(57) ABSTRACT

A method of measuring the surface flux of a component of a soil gas comprises placing a chamber for collecting said gas sealably in contact with a soil the chamber initially containing atmospheric gas. Then, the chamber is connected to a purging apparatus to purge the chamber of all measurable traces of the component. Then the soil is permitted to enter the chamber. The method uses a system for measuring the surface flux of the component. The system comprises a chamber having an open bottom for collecting soil gas, a venting port, an exhaust port having a valve and means to purge the chamber of the component.

### FIELD OF THE INVENTION

The present invention relates generally to the field of soil gas measurement. More specifically, the invention relates to a method and apparatus for measuring soil gas surface flux.

### BACKGROUND OF THE INVENTION

Global warming has recently led to an increased interest in measuring soil gas emissions, particularly emissions of carbon dioxide (CO₂), methane, nitrous oxide and other greenhouse gases. Both global change and local change may significantly affect soil carbon dioxide emissions. Such emissions have two primary sources: bacteria, fungi and other microbes that decompose organic matter (heterotrophic); and roots, mosses and other plants (autotrophic). Since the two sources may respond differently to various global and local effects, it is desirable to partition the two in order to make more reliable predictions about future emissions.

Stable isotopic tracers are used in many areas of terrestrial ecosystem science to shed light on rates or fates of physical or biogeochemical processes. Stable isotopes of carbon (C) and oxygen (O) are frequently used above ground in carbon cycling studies to separate photosynthetic assimilation and ecosystem respiration into their component fluxes, and in opportune circumstances using dual-isotope models to distinguish between ecosystem autotrophic and heterotrophic respiration. Below ground, laboratory studies using natural abundance δ13C and δ18O have been used successfully to isolate autotrophic and heterotrophic soil respiration.

Current commercial soil surface flux systems consist of measurement sensors and a microcomputer/datalogger coupled to an inverted headspace chamber placed on the soil surface. Over time, the increase in headspace concentration as a result of diffusion of gases out of the soil is measured and used to calculate soil surface flux.

Isotopic sampling of soil surface flux is typically done using static chambers, and Keeling plot analysis of several samples taken during a period of headspace equilibration, requiring an operator to monitor the sampling chambers. Error in the Keeling method can result from small ranges of concentrations used to extrapolate the y-intercept value for the isotopic signature of the mixing gas. Such extrapolation is required because the sampling chamber initially contains atmospheric air which includes detectable levels of the components to be measured in the soil gas.

Accurate partitioning of gas sources, such as autotrophic and heterotrophic carbon dioxide, using isotopic signatures requires resolution of relatively small differences, often in the order of 2 to 3 permil. Sample collection, handling and laboratory analysis typically is associated with reproducibility errors in the order of 0.2 to 0.3 permil. However, extrapolation of measured data in Keeling plot analysis can greatly increase the error. Even under the best circumstances, Keeling plot error is likely to be in the order of 0.2 to 0.3 permil, and in less than excellent circumstances, the error introduced from Keeling plot analysis can easily be as much as 2 permil.

The Keeling method also suffers from the disadvantage of requiring multiple measurements over a period of time sufficient to perform the extrapolation to estimate the isotopic signature of the soil gas alone.

Similar disadvantages are associated with techniques for measuring other soil gas components, such as radon and helium, not dependant on using isotopic signatures to partition sources.

There is therefore a need for a method that is simpler, requires less monitoring and is more precise than the current method of measuring soil gas flux. There is also a need for apparatus that facilitates implementing such a method.

### SUMMARY OF THE INVENTION

The present invention seeks to address or ameliorate the above described need, or at least to address or ameliorate one or more shortcomings or disadvantages associated with existing apparatus and methods for measuring soil gas surface flux.

In accordance with a first aspect of the present invention, there is provided a method for measuring surface flux of a component of a soil gas. The method includes the steps of: placing an open bottomed chamber for collecting the soil gas sealably in contact with the soil, the chamber initially containing atmospheric air; connecting the chamber to a supply of purging gas essentially free of the component of the soil gas to be measured; purging the chamber with sufficient purging gas to remove essentially all measurable traces of the component from said chamber; and after allowing sufficient time for an amount of said soil gas to flow into said chamber, collecting and analyzing at least one sample of said soil gas from said chamber.

Preferably, the volume of purging gas is at least approximately 10 times the volume of said chamber. Advantageously, the method includes the further step of measuring the concentration of the soil gas component contained in the chamber after allowing the soil gas to flow into the chamber. Most advantageously the purging step and the measuring step are controlled by an automated controller.

According to another aspect of the present invention, there is provided a system for measuring the surface flux of a component of a soil gas comprising a chamber for collecting such soil gas, means for supplying a purging gas to purge the chamber, and means for collecting a sample of the soil gas from the chamber. The chamber has an open bottom for sealably contacting the soil, and has a venting port and an exhaust port with a valve. The purging means supplies a purging gas essentially free from the component of the soil gas to be measured, and can purge the chamber with sufficient purging gas to remove essentially all measurable traces of such component.

Preferably, the purging means includes a pump for supplying air from the atmosphere and scrubbing means for removing the component from such supplied air. More preferably, the venting port is fitted with a venting hose which prevents atmospheric air from entering into the chamber during collection of the soil gas. Advantageously, the chamber further comprises a sampling port for taking samples of the soil gas from within the chamber. Most advantageously, the system further comprises control means for controlling the purging of the chamber.

According to yet another aspect of the present invention, there is provided a sampling chamber for collecting soil gas for use in measuring the surface flux of a component of such soil gas. The chamber comprises a receptacle suitable for containing a soil gas sample, having an open bottom adapted to be positioned sealingly to a soil sample, and having a venting port, a sampling port, and an exhaust port.

Preferably, the chamber includes a removable bottom section for sealably contacting said soil sample, and a cap section mating with said bottom section. Most preferably the sampling port includes a needle adapted to permit samples of soil gas collected in said chamber to be taken when an evacuated vile is operatively placed on the needle.

The invention allows for expanded measurement capabilities from automated soil surface flux systems. Soil gas flux data is increasingly being coupled to complex laboratory gas measurements in environmental research, and where practical, important gains may be realized in terms of hardware and labor costs, measurement and analysis time by integrating flux measurements and isotope sampling together. The accuracy of the sample collection technique is improved by the present method, in addition to traceability, since field samples are usually collected manually.

This method provides a simple, fast and effective alternative to current methods of measuring components of soil gas surface flux, such as carbon dioxide and other greenhouse gases, or radon or helium, where constant attention to chamber sampling is not required during equilibration.

### BRIEF DESCRIPTION OF DRAWINGS

These and other features of the present invention will become more apparent from the following description in which reference is made to the appended drawings wherein:

FIG. 1 shows a view of the opened sampling chamber in accordance with an embodiment of the present invention;

FIG. 2a is an perspective view of the of the chamber of FIG. 1;

FIG. 2b is an perspective bottom view of the cap of the chamber of FIG. 1;

FIG. 3 is a view of the chamber of FIG. 1 connected to a purging apparatus;

FIG. 4 is a view of a portable test equipment in accordance with another embodiment of the present invention;

FIG. 5 is an isometric view of apparatus components in accordance with another embodiment of the present invention;

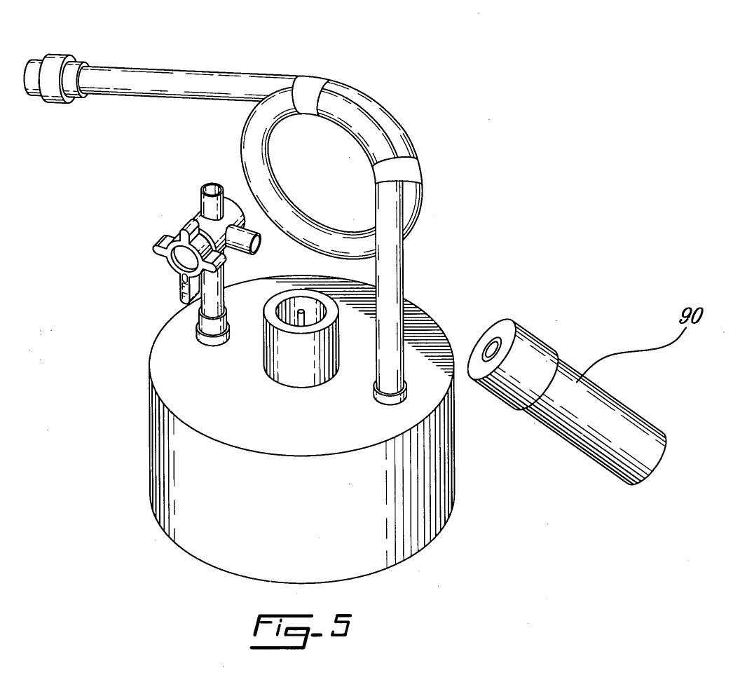

FIG. 6 is a flowchart of a process in accordance with an embodiment of the present invention;

FIG. 7 is a perspective view of a chamber in accordance with another embodiment of the present invention;

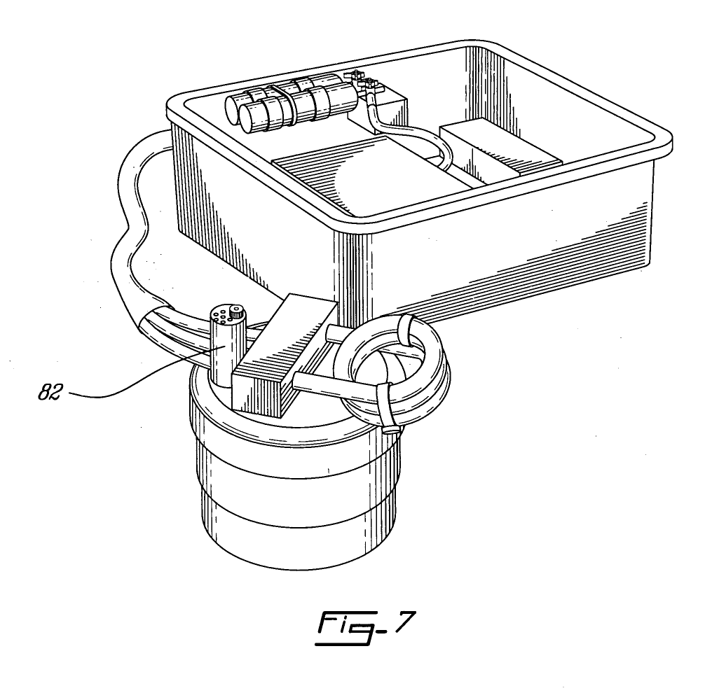

FIG. 8 is a view of apparatus components in accordance with another embodiment of the present invention.

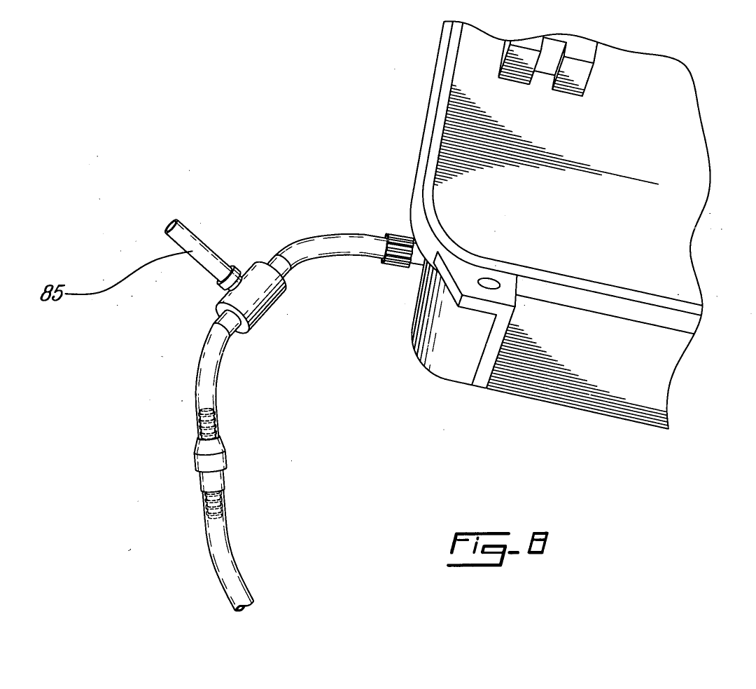

### DETAILED DESCRIPTION OF THE INVENTION

Automated soil gas surface flux measurement systems are relatively new to the commercial marketplace and are used to quantify rates of greenhouse gas release from soils. The present invention enhances the flexibility of these systems by providing a means to execute the controlled collection of uncontaminated samples for laboratory analysis. These improvements are ideal for the collection of soil gas samples for Isotope Ratio Mass Spectrometry, as isotopic and flux data are increasingly being coupled in soil flux research.

### Apparatus Design

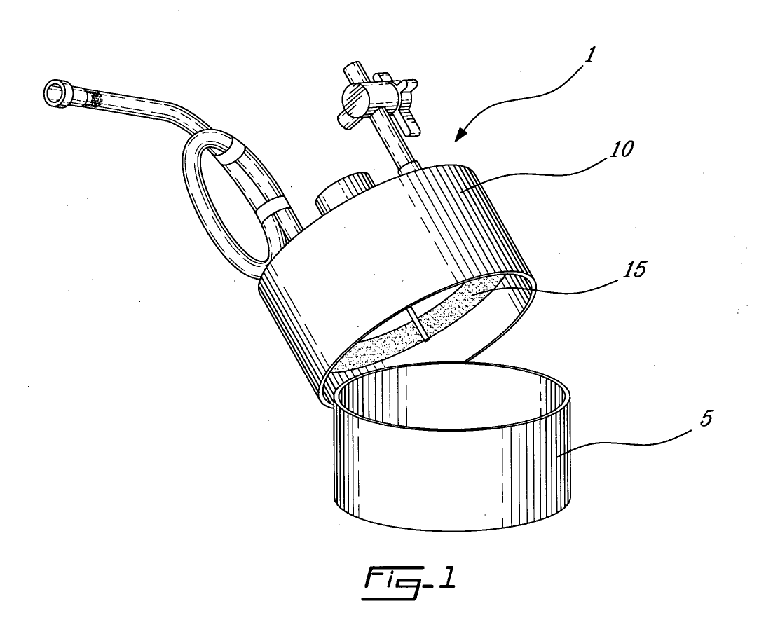

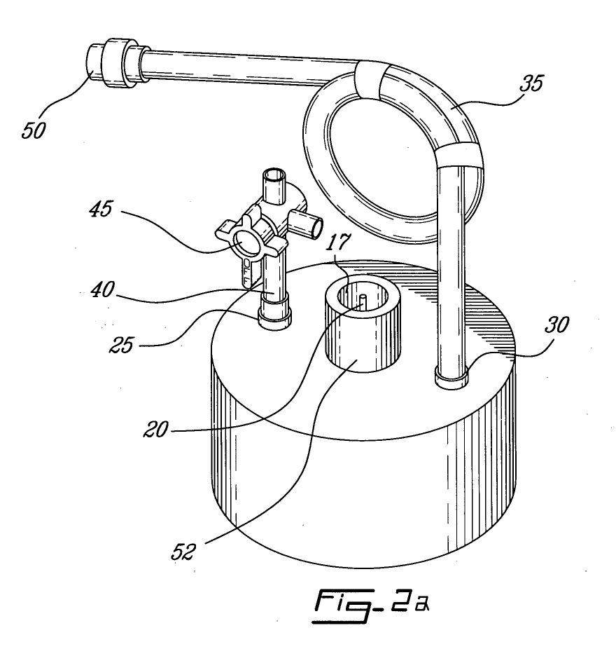

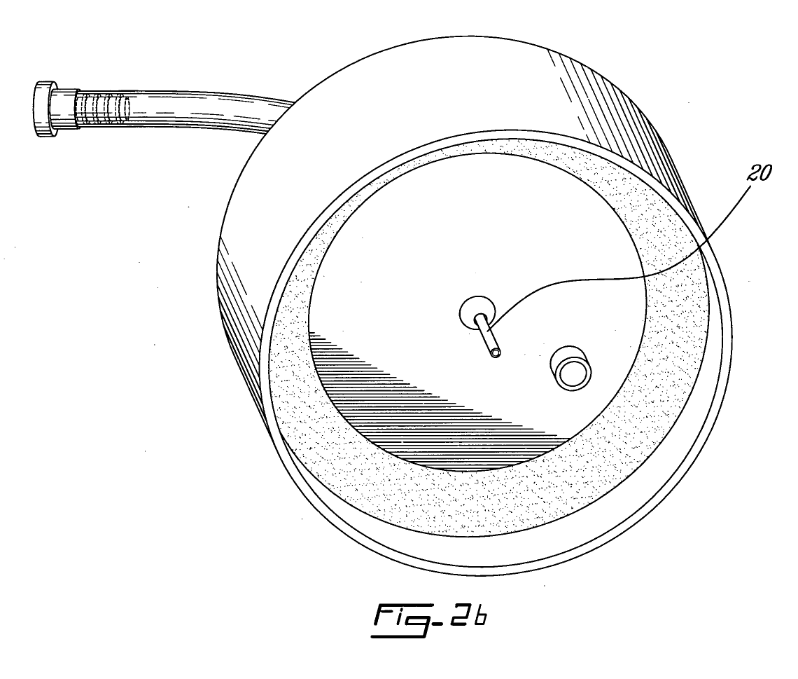

As shown in FIG. 1, the sampling chamber 1 comprises a field collar 5 and a cap 10. The inside circumference of the cap 10 has a closed cell foam-sealing strip 15 to ensure a proper seal onto the field collar 5. The field collar 5 is a cylinder with both ends open and is adapted to receive the cap 10. As shown in FIG. 2a, the top of the cap 10 is adapted to receive, in a sampling port 17, a double-ended tubular needle 20 that extends through the top of the cap 10 into the headspace 22 of the chamber 1. FIG. 2b shows the needle protruding into the interior of the chamber 1. The top of the cap 10 is also fitted with an exhaust port 25 and a venting port 30 that doubles as a purging inlet. A venting hose 35 is connected to a venting port 30 while an exhaust hose 40, fitted with an exhaust valve 45, is connected to an exhaust port 25. The chamber 1 is approximately 100 mm I.D.×50 mm tall, offering a relatively high surface area to volume ratio. The needle 20, and both the venting hose 35 and the exhaust hose 40 are respectively incorporated into the chamber 1 for sampling, pressure equilibration, and purging.

The design of a suitable collection chamber should address many considerations such as volume, effective mixing, wind protection, venting, and collar installation. Because soil CO₂ is typically present in relatively high concentrations and drives a strong concentration gradient outwards from the soil, a wide variety of chamber designs are functionally acceptable, except large chamber volumes in which mixing may become an issue. The present chamber 1 is relatively small. Mixing in such a small chamber is rapid, helping to obtain representative headspace samples. Wind protection is provided by the foam-sealing strip 15 and ensures a tight fit with the field collar 5. A wind deflector 52 is also fitted around the needle 20. This is important because the δ13C content of atmospheric CO₂ is considerably different from that of soil respired CO₂. Because wind can cause pressure variations within the chamber 1, wind protection and venting are important to ensure that the chamber 1 is properly characterizing the isotopic composition of the CO₂ emissions. Venting is critical in controlling pressure variations due to the collection of gas within chamber 1 and consequently, the size of the venting hose 35 is selected to ensure that there is no diffusion of atmospheric air into the chamber 1 during the collection process. Testing has revealed that a venting hose 35 of 2 to 3 mm I.D. and 400 mm in length is adequate to ensure that no back diffusion occurs during sampling. It is also important to ensure that the field collar 5 is installed without gaps or improper seals with the soil to prevent atmospheric invasion during sampling.

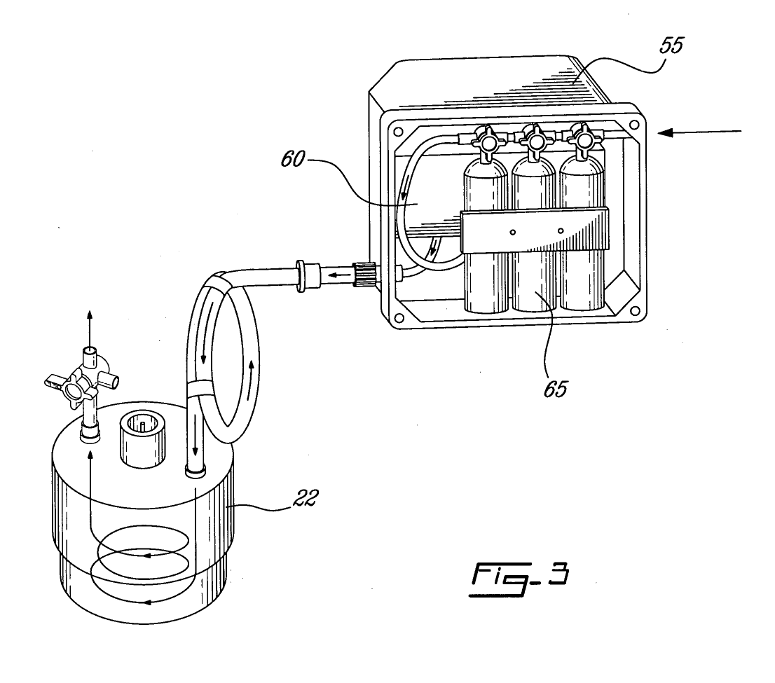

A unique characteristic of the chamber 1 is its ability to be purged in-situ. A quick-connect connector 50 is installed at the end of venting hose 35, which can be opened temporarily and connected to a purging apparatus 55, as shown in FIG. 3. The purging apparatus 55 uses a small pump 60 and filtering columns 65 containing a chemical that removes CO₂ from the air. It is possible to use soda lime, for example, in the columns 65 to remove the CO₂ from the air. The pump 60 draws air from the free atmosphere, filters it through the filtering columns 65 and produces CO₂-free air that is forced in to the chamber 1 and used as a purge gas. Alternatively, an inert gas could be used as the purge gas and introduced in the chamber 1 either through the pump 60 or through a pressurized source of such inert gas.

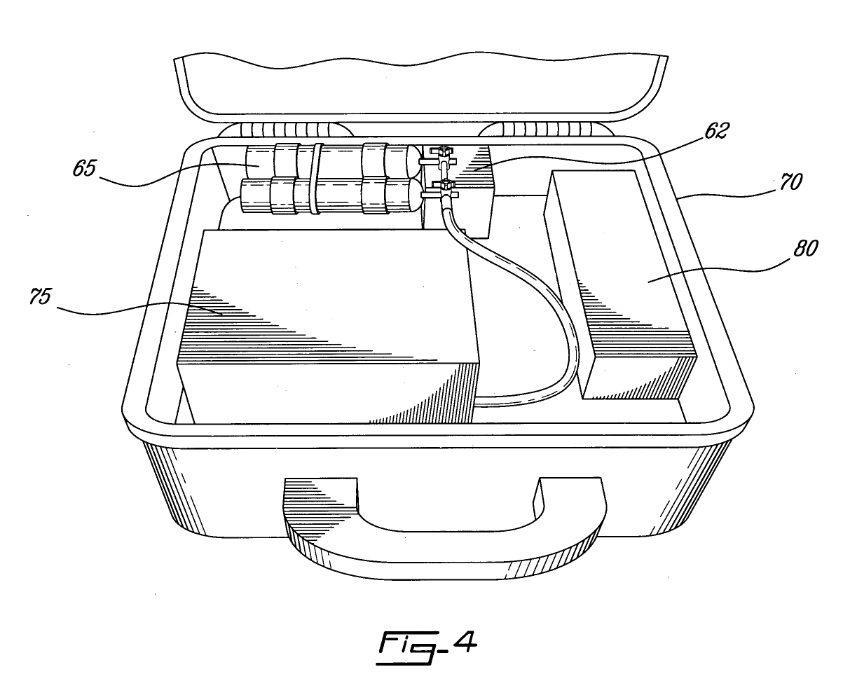

In another embodiment of the invention, the chamber is connectable to portable test equipment 70 as shown in FIG. 4. The portable test equipment 70 comprises a CO₂ analyzer 75, a purging apparatus 55, a loop pump 62 and a controller 80. The chamber 1a includes a check valve 82 that closes when there is no purging flow coming from the test equipment 70. Since CO₂-free air is sent from the portable test equipment 70, there has to be a way to stop any leakages into the chamber when purging apparatus 55 of portable test equipment 70 is not on. The controller 80 is programmed to, on command, completely purge the chamber 1a of CO₂, measure the CO₂ flux from the soil and warn when the CO₂ concentration is sufficiently high to allow the sample to be drawn via the sampling port 17. The loop pump 62 circulates the air from the chamber to the CO₂ analyzer 75 and then back to the chamber. The sampling port 17 consists of a tubular needle 20 extending into the continuous gas flow, with removable sealed cap 85 to isolate the automated system's gas flow loop until samples are drawn via the needle 20 into evacuated vials 90. The needle may be placed in the chamber 1a or in a hose 35 or 40.

### Method

In existing methods, a Keeling plot is used to predict the isotopic signature of one gas moving into another. A flux chamber similar to chamber 1 is deployed on the soil surface initially with atmospheric air in it. The CO₂ in this air has a carbon isotope “signature” of −8 permil. Because there is a relatively large amount of CO₂ in soil compared to that in atmospheric air, CO₂ moves from the soil into the chamber as diffusion tries to equalize this imbalance. It is possible to measure the change in isotopic signature relative to time or, by the Keeling plot approach, relative to the inverse of the concentration increase in the chamber.

The method disclosed herein purges the CO₂ contained in atmospheric air inside the chamber before the soil CO₂ is allowed to fill it. There is therefore no need to use the Keeling method and hence avoids errors associated with mathematical extrapolation.

In nature, CO₂ moves out of soil because there is a larger concentration of it in the soil than there is in the atmosphere. For example, CO₂ in the atmosphere is about 370 ppm while CO₂ in soil ranges between 1000 to 40,000 ppm. The speed with which the CO₂ moves out is proportional to the difference in concentration. When a collection chamber is set on the soil surface, it accumulates CO₂ that is moving out of the soil. As it accumulates, the soil CO₂ flux rate slows down, because the difference in the concentration gradient across the soil surface reduces.

To eliminate all of the CO₂, it is preferable to purge about 10 volumes of CO₂-free air through the chamber, (that is, a 1000% purge). This eliminates essentially all measurable traces of CO₂ from the initial atmospheric air in the chamber. Thus if the chamber is 500 ml, 5000 ml of purging air is preferably used. This air is manufactured by “scrubbing” CO₂ from atmospheric air. By experience, it has been found that a 1000% purge provides reliable results; however, somewhat less purging volume may be acceptable in some circumstances.

The method of the present invention uses a high volume pump 60 to force the CO₂-free air into the chamber 1 and out the exhaust port 25. When using the portable test equipment 70, a smaller loop pump 62 is connected to the chamber 1. The high volume pump 60 may be left connected to the chamber 1 at all times by using the check valve 82 which isolates the air in the venting hose 35 when it is not in use such that air does not leak into the chamber 1. While purging, the loop pump 62, which circulates the air from the chamber 1 to the CO₂ analyzer 75 and back to the chamber 1, is kept running at the same time in order to monitor and log concentrations and to clear out the CO₂ analyzer and associated tubing. All this is done automatically.

With this automated system that uses the portable equipment 70, it is preferable to try to keep the “natural” concentration gradient in the middle of the concentration range. The system has an alarm that goes off when chamber 1 concentrations reach approximately 750 ppm or about double the atmospheric concentrations. When the alarm sounds, the measurement is over, a user may take his isotopic sample from the needle, and move on. The concentration gradients favor fast CO₂ movement into the chamber 1 when concentrations are below atmospheric, and slower movement when above. These offset one another, so standard rules, which is to have atmospheric concentrations as the mean of those seen by the CO₂ analyzer 75 during the measurement period, are used. In this case, a wider range of concentrations is used. The other benefit of the present invention is that typical isotope machines like to be bathed luxuriously in CO₂, above 700 ppm.

The chamber is squat relative to the prior art designs so that concentrations can build up very quickly. As a result, a CO₂ flux measurement and isotope sampling takes only about 5 to 15 minutes depending on the soil CO₂ emission rate. The first 40 seconds is spent purging. The known prior art chambers are about 5 times as high as the present chamber, so it would take 5 times longer to fill the prior art chamber. The prior art methods measure over very small ranges of concentration relative to the accuracy/precision of the CO₂ detector used.

In the method of the present invention, the field collar 5 is pushed approximately 40 mm into the soil at the sampling site prior to the sampling period. It is important to ensure that the field collar 5 is installed well with no gaps or improper seals with the soil. The field collar 5 should preferably be installed at least 24 h prior to sampling to eliminate any re-equilibration error. Indeed, installing the collar in the soil causes disturbance resulting in increased CO₂ emissions for a while. After this waiting period, the cap 10 is slid onto the field collar 5 in order to create a headspace 22 in which the soil gas is collected.

As mentioned before, a characteristic of the chamber 1 is its ability to be purged in situ. Advantageously, the Keeling plot and multiple sample methodology are not required when the headspace 22 of chamber 1 starts the equilibration period completely free of atmospheric CO₂. This is very useful for relatively small chambers.

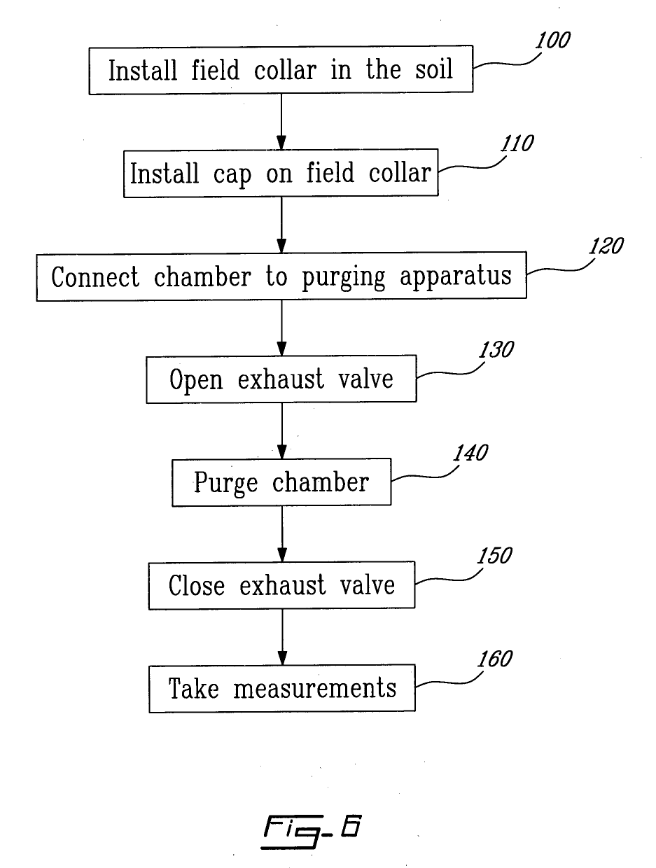

As shown in FIG. 6, the chamber 1 is connected to the purging apparatus 55 by connecting the venting hose 35 to the purging apparatus (step 120). The exhaust valve 45 is then opened to allow air with CO₂ to exit (step 130). The purging process 140 is performed prior to taking measurements 160. The exhaust valve 50 connected to the exhaust hose 40 may be opened and closed at will. The purging process 140 consists in connecting the pump 60 to the quick-connect connector 50, pumping air through the soda lime columns and into the headspace 22. During the purging process 140, the exhaust valve 45 is left open to allow free flow of the CO₂-free air into and out of the headspace 22. When the purging 140 is complete, the exhaust valve 45 is closed (step 150) and the pump 60 may be disconnected if required. The headspace 22, now with zero initial CO₂ concentration, is then allowed to collect soil gas emissions. Purging the chamber with approximately 10 times the headspace volume of CO₂-free air ensures that the samples collected will not be contaminated with atmospheric CO₂ that may have been trapped in the headspace 22 when the chamber and the collar were initially coupled.

Alternatively, it is possible to purge the headspace 22 from CO₂ by connecting the chamber 1 to the purging apparatus 55 in a closed loop. In this case, the exhaust hose 40 is connected to an inlet of the purging apparatus 55 which, instead of filtering ambient air and pumping it in the chamber 1, simply filters the air that is already trapped in the chamber 1 and the hoses 35 and 40 until there is no CO₂ left in the air.

Alternatively to performing the purging step manually, instructions to that end may be incorporated directly into the software of controller 80, such that this step is performed automatically. Software instructions include a full purge (or scrub) of atmospheric gases from the chamber 1, after which the soil surface gas flux is allowed to fill the chamber 1 and the system can perform its normal measurement routine.

After a given period of time, samples are drawn via the sampling port 17 into vials 85. The vials 85 are evacuated test tubes having no air inside. When their bottom is pierced with the tubular needle 20, a sample of gas is drawn out of the chamber into the vial 85. The sample may then be analyzed for isotopic content. Alternatively, this could be performed by a portable test equipment 70 directly on site if fitted with the right testing equipment for analyzing the isotope content.

### Laboratory Testing

Since the chamber 1 recruits soil CO₂ by diffusion, it likely initiates a fractionation of ambient soil δ13CO₂ values. Assuming an infinite source (soil) and sink (chamber), this fractionation will be −4.4 permil, or the theoretical fractionation associated with CO₂ diffusion. However, given non-infinite sources and increasing equilibration time, δ13CO₂ will increasingly catch up with faster moving δ12CO₂, reducing the apparent theoretical fractionation. To characterize the performance of the chamber and technique in relation to these parameters, repeatability and fractionation tests were performed in laboratory columns to test the effects of equilibration time, purge time, and soil diffusivity on observed isotopic fractionation values.

The column incorporated a lower reservoir. The reservoir was an artificial soil composed of washed silica sand packed to 40% porosity. This artificial soil was held between metal screens and a collar at the top of the column to which the flux chamber could be fitted. A gas mixture from a low pressure air cylinder was introduced, via a valve, into a reservoir at the bottom. Prior to each experiment, the chamber was installed on top and the entire system (column and chamber) was flushed with approximately 10 liters of a CO₂/N₂ mixture introduced into the bottom reservoir. This purge flow ultimately exited by the flux chamber exhaust port 25, ensuring a uniform concentration and δ13CO₂ throughout the reservoir, column and chamber. The δ13CO₂ of the gas mix was roughly −1 permil depending on the batch. After purging the system with this known gas mixture, the chamber headspace 22 was purged of CO₂ according to our sampling protocol. After a headspace equilibration period, isotopic samples were drawn from the chamber and the reservoir in 10 ml evacuated vials 90.

The first test, performed at zero percent soil moisture, was used to determine repeatability and fractionation associated with variations in headspace equilibration time. In this test, repeated measurements were performed using headspace equilibration intervals of 5, 10 and 15 minutes. The second test held equilibration time constant at 10 minutes, but effects of volumetric water content was tested using volumetric water contents of 0, 10 and 30 percent. In the final lab test at zero percent soil moisture, purge times of 20, 40 and 80 seconds (equivalent to 5, 10 and 20 chamber volumes) were used to examine fractionation effects. For all tests, a minimum of five replicate tests were performed to characterize repeatability.

### Field Testing

Using the sampling method, the importance of physical determinants on forest-floor δ13CO₂ flux variability relative to variability resulting from biological factors was assessed.

Using the model of Cerling et al. (1991), it was sought to assess the maximum potential range of isotopic effects that could be expected from variations in atmospheric invasion across the sampling grids. Soil gas diffusivity and respiration rate are the primary determinants of atmospheric invasion, and potential site δ13CO₂ spatial variability related to physical mechanisms. Soil respiration rates were calculated for each sampling point from IRMS concentration data, and for LV1 they compared favorably with a Licor 8100 survey that had been conducted 10 days earlier. Spatial variability in respiration rate from the Licor and those calculated from the isotopic chambers were comparable at σ=1.12 (n=20) and σ=0.76 (n=40), respectively. Unlike respiration rates, simultaneous collar-specific moisture data was lacking. An approximation of forest floor moisture variability from gravimetric bulk density analysis of soil cores done in September 2004 was obtained, when TDR measured soil moisture values at site meteorological stations were only 1% different than during this study. These showed an average soil moisture content (n=20) of 13% with a standard deviation of 7% across all micro-topography. These moisture values were used to calculate soil CO₂ diffusivity rates with the model of Millington (1959) using 40% total soil porosity (sand) and were applied to all sites under the assumption that they may all show similar variations in soil moisture because of similar mound and hollow micro-topography. The permil offsets predicted by the Cerling et al. (1991) model were averaged over the top 5 cm of soil, since this was a typical observed recruitment depth under the range of experimental laboratory conditions. The average isotopic offsets were calculated for two scenarios that illustrate the maximum (lowest site respiration, highest site diffusivity) and minimum (highest site respiration, lowest site diffusivity) potential effects of atmospheric invasion. The source and atmospheric signatures for this simulation were estimated at −27 and −9 permil, respectively.

Forest floor variability resulting from biological factors, in particular the isotopically distinct contributions from roots and microbial activity, was assessed using radial distance from trees as a proxy for the spatial distribution of root and microbial sources. Recent studies highlight relationships between tree proximity and overall flux magnitude, suggesting that isotopic signatures of soil surface flux may vary similarly across the forest floor as a result of predictable source spatial variation. Tree proximity was measured for each grid sampling location. The number of trees within a radius of 1.5 m was also enumerated.

The field tests were conducted at five forested sites in Atlantic Canada. Four parallel rows of collars were installed, each 10 m long with 1 m collar spacing. Rows were separated by 2 m, providing a sampling grid with 40 points, covering a forest floor surface area of 80 m². Collars were pushed approximately 40 mm into the soil at the sampling site prior to the sampling period. The chamber equilibration period at three sites (LV1, LV2 and P2) was 10 minutes whereas equilibration period at the two other sites (NFF and NFS) was 20 minutes to compensate for smaller CO₂ fluxes at these sites. Samples from the headspace were collected via the needle in 10 ml evacuated vials and were analyzed within 24 hours to minimize the likelihood of contamination. Repeatability and microscale variability were also tested in the field by, firstly, repeatedly purging and sampling from the same chamber and, secondly, installing many chambers beside one another in treeless areas where spatial variability was assumed to be minimal.

### Mass Spectrometry

Both field and lab gas samples were analyzed on a continuous flow stable isotope mass spectrometer (GV Instruments) and δ13C values are reported as per mil variation from the δ13C isotopic standard Pee Dee Belemnite:

$$δ13 C = \frac{{R}_{sample} - {R}_{std} \times 1000}{{R}_{std}}$$

where Rₛₐₘₚₗₑ and R_{std} are the δ13C/δ12C ratios of the sample and the standard, respectively. During analysis of both field and laboratory gas samples, the mass spectrometer was found to have a error of <0.2 per mil between replicate samples. Concentrations of bulk CO₂ were determined simultaneously.

### Laboratory Repeatability and Fractionation

Reproducibility was high for all laboratory measurements, and added only small error beyond the analytical variability error with IRMS analysis. The maximum observed standard deviation (σ) of replicate column experiments (n=5) was 0.31 permil, associated with shorter headspace equilibration time intervals of 5 minutes. As equilibration time increased to 10 minutes or beyond, standard deviations among replicate column experiments fell to roughly 0.20 permit regardless of soil gas diffusivity/column soil moisture content or other parameters that were manipulated during the laboratory tests. Standard deviations for all tests and experimental conditions (n=60) were 0.24 permil, which compares favorably with Keeling approaches, where propagation of R2 uncertainly in surface flux chamber data results in additional errors beyond reproducibility and IRMS precision. The present method of isotopic flux sampling proved to be reliable, and contributes only small errors beyond those associated with IRMS analysis, despite several opportunities for contamination common to all flux chamber methods, including chamber venting, chamber sealing, vials purging, vacuuming and leakage before analysis. Through careful method design and laboratory practices, these opportunities for error have been minimized, and are not appreciable individually or collectively.

Fractionation associated with the sampling method varied according to headspace equilibration time. Short equilibration times maximize the fractionation associated with the method, where an offset close to the theoretical −4.4 permil offset was observed. Offsets decrease linearly with increasing equilibration time. Paired samples t-tests reveal that the observed variability in fractionation at different equilibration times cannot be explained by the repeatability error associated with the various trials. This time dependent fractionation is understood as being the product of the non-infinite isotopic source (soil) and sink (chamber) system. Headspace concentrations accumulate quickly within the chamber initially, and slow down as the soil-chamber concentration gradient weakens, which allows for increased equilibration of slow-moving δ13CO₂. Headspace equilibration time is held constant for site grid surveys, so relative isotopic values within site are preserved but care must be exercised when comparing across sites. For cross-site comparisons where equilibration times must be tuned according to the magnitude of expected soil respiration values, equilibration times must be taken into account.

The degree of fractionation associated with the sampling method showed a strong apparent dependence on soil gas diffusivity, which was varied by changing the water content of the column soil. When water was introduced into the system, error was unaffected, but fractionation under 10% and 30% water contents was approximately half of that observed under dry (0%) conditions, approximately 2 permil. These water content of 0%, 10% and 30% are equivalent soil gas diffusivities of 4.62×10−6 m²/s, 1.77×10−6 m²/s and 4.56×10−8 m²/s respectively. This diffusivity-fractionation effect was further investigated by adding three extra sampling needles in the experimental column, from which the soil profile CO₂ concentrations were measured. For dry soils, CO₂ is recruited from deeper in the profile, whereas in wet soils, near-surface CO₂ is preferentially exploited to increase chamber concentrations, despite the lower gas-filled pore space and more limited CO₂ pool. Average depth recruitment over all tests was 5 cm. Our three point concentration profile means that localized recruitment effects near the surface may have potentially been missed, but it is speculated that in wet soils, shallow recruitment serves to limit the apparent size of the source. Hence, in effect, δ13CO₂ and δ12CO₂ has more opportunity for equilibration. Regardless of the cause, since fractionation was stable at approximately −2 permil over the wide range of soil moisture/diffusivity values observed at all sites, the diffusivity-related sampling fractionation can be assumed to be invariant for the purposes of the study.

In completely dry soils, trends were observed between purge time and measured isotopic values. At 40 s or longer purge times (equivalent to 10 chamber volumes), fractionations were stable at almost −4.4 permil. However, during short (20 s) purge times, only approximately 2 permil fractionation was observed, or approximately half that of the 40 and 80 second purge times and theoretical diffusion fractionation. The assumed mechanism for these purge time fractionation discrepancies is the concentration gradient induced during purging, which may result in infiltration of the CO₂-free air into the sand matrix. This effect can definitely not be explained by residual atmospheric δ13CO₂ contamination in the chamber, which would instead serve to further deplete signatures beyond the theoretical −4.4 permil. But, again, for the purposes of the study, purge time fractionations effect are unimportant, because purge times were always held constant in the field. In summary, the cumulative errors associated with sampling and analysis using the present method of flux sampling are small (0.2-0.31 permil), and are only modestly higher than the accepted ˜0.2 permil error associated with CF-IRMS analysis. In addition to speed, the present method may be associated with less sampling variability than Keeling plot approaches, especially where headspace concentration increases are small due to low flux rates. Laboratory studies indicate that fractionations are associated with this sampling method, serving to offset the measured isotopic value from the actual δ13CO₂ signature in the soil. Fractionations were lower for low soil gas diffusivities (high moisture content), long equilibration times, and shorter purge times. For the purposes of field study, sampling fractionations can be held constant at roughly −2 permil with careful selection of method parameters. The low variability and speed associated with this technique boosts the effectiveness of root/microbial partitioning, because measurement variability can be kept low and many replicate δ13CO₂ samples can be taken in a short span of time. Aside from source signature difference, spatial replication and low variability are critical parameters in source partitioning studies.

It will of course be appreciated that many modifications and alternative embodiments are possible within the broad scope of the present invention. For example, the gas sampling methodology explained herein may also be applied to the measurement and sampling of other soil source gases such as methane or nitrous oxide, for which automated soil surface flux systems may not yet be commercially available. Additionally, the invention may provide a means for collecting surface flux samples for analyses other than isotope ratio mass spectrometry, where contamination by atmospheric air is undesirable. Obviously, with the right purging apparatus, many components other than CO₂ may be purged from a gas.

The present invention has been described with regards to preferred embodiments. The description as much as the drawings, were used to help the understanding rather than to limit the scope of the invention. It will be obvious to one skilled in the art that several modifications or variations may be brought to the invention without departing from the scope of the invention as described herein and are intended to be covered by the present description.

I claim:

1. A method of measuring the surface flux of a component of a soil gas comprising the steps of:
placing an open bottomed chamber for collecting said gas sealably in contact with a soil location, said chamber initially containing atmospheric air;
connecting said chamber to a supply of purging gas essentially free of such component;
purging said chamber with sufficient purging gas to remove essentially all measurable traces of such component from said chamber; and
after allowing sufficient time for an amount of said soil gas to flow into said chamber, collecting and analyzing at least one sample of said soil gas from said chamber.

2. A method as defined in claim 1, wherein the volume of purging gas used is at least approximately 10 times the volume of said chamber.

3. A method as defined in claim 2 further comprising the steps of opening an exhaust valve before purging and closing it after the purging is completed.

4. A method as defined in claim 3 wherein the step of collecting said at least one sample comprises using an evacuated vial.

5. A method as defined in claim 4 further comprising the step of measuring the concentration of said component in said soil gas contained in said chamber.

6. A method as defined in claim 5 further comprising the step of controlling said measuring step using an automated controller.

7. A method as defined in claim 6 further comprising the step of controlling said purging step using an automated controller.

8. A system for measuring the surface flux of a component of a soil gas comprising:
a chamber having an open bottom for sealably contacting a soil location and collecting such soil gas, said chamber having a venting port, and an exhaust port with a valve;
means for purging said chamber with sufficient purging gas to remove essentially all measurable traces of such component from said chamber; and means for analyzing such soil gas collected in said chamber after purging.

9. A system as defined in claim 8 further comprising:
a bottom section for sealably contacting said soil at a first end of said bottom section;
a cap for sealing a second end of said bottom section.

10. A system as defined in claim 9 wherein said purging means can supply a purging gas essentially free of such component in a volume at least approximately 10 times the volume of said chamber.

11. A system as defined in claim 10 wherein said purging means includes a pump for supplying a purge gas, and scrubbing means for removing such component therefrom.

12. A system as defined in claim 11 wherein said purge gas is an inert gas free of the component.

13. A system as defined in claim 11 further comprising a seal between said cap and said bottom section.

14. A system as defined in claim 13 wherein said exhaust port comprises a valve.

15. A system as defined in claim 14 wherein said venting port is fitted with a venting hose, said venting hose preventing atmospheric air from entering into said chamber during collection of said soil gas.

16. A system as defined in claim 15 wherein said chamber further comprises a sampling port for taking samples of said soil gas inside said chamber.

17. A system as defined in claim 16 wherein said sampling port further comprises a needle passing through said cap for fluid communication between said chamber to an exterior of said chamber.

18. A system as defined in claim 17 wherein said sampling port comprises means to protect said needle from a wind.

19. A system as defined in claim 18 wherein said needle is adapted to let samples of soil gas go through when an evacuated vial is operatively placed on said needle.

20. A system as defined in claim 19 further comprising a controller connected to said purging means for controlling a purge cycle.

21. A system as defined in claim 20 wherein said exhaust port is connected to said purging means.

22. A system as defined in claim 21 wherein said needle extends into said venting hose.

23. A system as defined in claim 22 wherein said needle is fitted with a removable sealed cover to isolate said needle from atmospheric air.

24. A system as defined in claim 23 wherein said needle is adapted to let samples of soil gas go through when an evacuated vial is operatively placed on said needle.

25. A system as defined in claim 24 wherein said purge cycle is complete when said component of said soil gas has been purged sufficiently from said atmospheric gas that no mathematical correction to a measurement is required.

26. A sampling chamber for collecting soil gas comprising:
a receptacle suitable for containing a soil gas sample, having an open bottom adapted to be positioned sealingly to a soil sample, and having a venting port, a sampling port, and an exhaust port.

27. A chamber as defined in claim 26 further comprising:
a removable bottom section for sealably contacting said soil sample; and
a cap section mating with said bottom section.

28. A chamber as defined in claim 27 further comprising a seal between said sections.

29. A chamber as defined in claim 28 wherein said sampling port further comprises a needle passing through said cap for fluid communication between said chamber to an exterior of said chamber.

30. A chamber as defined in claim 29 wherein said needle is adapted to let samples of said soil gas go through when an evacuated vial is operatively placed on said needle.

31. A chamber as defined in claim 30 wherein said exhaust port is fitted with a valve.

32. A chamber as defined in claim 31 wherein said venting port is fitted with a venting hose, said venting hose preventing atmospheric air from entering into said chamber during collection of said soil gas.

33. A chamber as defined in claim 32 wherein said sampling port comprises a wind deflector.

34. A chamber as defined in claim 33 wherein said needle is fitted with a removable sealed cover to isolate said needle from atmospheric air.
